(19) United States
(12) Patent Application Publication
(10) Pub. No.: US 2023/0293544 A1
(43) Pub. Date: Sep. 21, 2023

(54) METHODS, DOSAGE REGIMENS, AND COMPOSITIONS FOR TREATING HIDRADENITIS

(71) Applicant: PFIZER INC., New York, NY (US)

(72) Inventors: Brian Gerstenberger, Cambridge, MA (US); Andrew Fensome, Harvard, MA (US); Dafydd Owen, Concord, MA (US)

(73) Assignee: PFIZER INC., New York, NY (US)

(21) Appl. No.: 18/180,472

(22) Filed: Mar. 8, 2023

Related U.S. Application Data
(60) Provisional application No. 63/320,913, filed on Mar. 17, 2022.

(51) Int. Cl. A61K 31/55; A61P 17/02

(52) U.S. Cl. CPC A61K 31/55; A61P 17/02

(57) ABSTRACT

There are methods, dosage regimens, and compositions for treating hidradenitis suppurativa.

### CROSS-REFERENCE TO A RELATED APPLICATION

The present application claims priority based on U.S. Provisional Application No. 63/320,913, filed Mar. 17, 2022, the disclosure of which is incorporated herein in its entirety.

### FIELD OF THE DISCLOSURE

The present disclosure provides methods, dosage regimens, and compositions for treating hidradenitis suppurativa.

### BACKGROUND OF THE DISCLOSURE

Protein kinases are families of enzymes that catalyze the phosphorylation of specific residues in proteins, broadly classified into tyrosine and serine/threonine kinases. Inappropriate kinase activity, arising from mutation, over-expression, or inappropriate regulation, dys-regulation or de-regulation, as well as over- or under-production of growth factors or cytokines has been implicated in many diseases, including but not limited to cancer, cardiovascular diseases, allergies, asthma and other respiratory diseases, autoimmune diseases, inflammatory diseases, bone diseases, metabolic disorders, and neurological and neurodegenerative disorders such as Alzheimer's disease. Inappropriate kinase activity triggers a variety of biological cellular responses relating to cell growth, cell differentiation, cell function, survival, apoptosis, and cell mobility implicated in the aforementioned and related diseases.

Thus, protein kinases have emerged as an important class of enzymes as targets for therapeutic intervention. In particular, the JAK family of cellular protein tyrosine kinases (JAK1, JAK2, JAK3, and Tyk2) plays a central role in cytokine signaling (Kisseleva et al., Gene, 2002, 285, 1; Yamaoka et al. Genome Biology 2004, 5, 253). Upon binding to their receptors, cytokines activate JAKs, which then phosphorylate the cytokine receptor, thereby creating docking sites for signaling molecules, notably, members of the signal transducer and activator of transcription (STAT) family that ultimately lead to gene expression. Numerous cytokines are known to activate the JAK family. These cytokines include the interferon (IFN) family (IFN-alpha, IFN-beta, IFN-omega, Limitin, IFN-gamma, IL-10, IL-19, IL-20, IL-22), the gp130 family (IL-6, IL-11, OSM, LIF, CNTF, NNT-1/BSF-3, G-CSF, CT-1, Leptin, IL-12, IL-23), gamma C family (IL-2, IL-7, TSLP, IL-9, IL-15, IL-21, IL-4, IL-13), IL-3 family (IL-3, IL-5, GM-CSF), single chain family (EPO, GH, PRL, TPO), receptor tyrosine kinases (EGF, PDGF, CSF-1, HGF), and G-protein coupled receptors (AT1).

Hidradenitis suppurativa (HS) is a chronic, inflammatory, recurrent, debilitating skin disease that usually presents after puberty with painful, deep-seated, inflamed lesions in the apocrine gland-bearing areas of the body. Zouboulis, C., et al., Dermatology, 231(2), pp. 184-190 (2015). HS presents a variable clinical course. One of the main features of the disease is the intertriginous occurrence, although other areas of skin may also be affected. The affected areas are, in decreasing order of frequency: inguinal, axillary, perineal and perianal as well as the submammary and/or intermammary fold in women, buttocks, mons pubis, scalp, area behind the ears and eyelids.

The prevalence of self-reported disease is about 1% in Western Europe. The average interval from the onset of symptoms to diagnosis is 7.2 years. Women are affected 2 to 5 times as frequently as men, and the disease may be more common in blacks than in whites. Disease severity ranges from mild (localized lesions) to severe (multiple areas of widely dispersed lesions, including interconnected sinus tracts and hypertrophic scars). Pain, drainage, and range of motion limitations from scarring can decrease the quality of life. Jemec, G. B., New Eng. J. Med., 366: 158-64 (2012); Kimball, A., et al., New Eng. J. Med., 375(5), pp. 422-434 (2016).

Pain is a prominent feature of HS, which is reflected in the recently defined set of core outcomes to be assessed in future trials. Thorlacius, L., et al., Brit. J. Derm., 179(3), pp. 642-650 (2018). The majority of patients rated their pain on a Numerical Rating Scale-11 (NRS 11) ranging from 4/10-10/10 and described it at various times as hot, burning, pressure stretching, cutting, sharp, taut, splitting, gnawing, pressing, sore, throbbing, and aching. Currently adalimumab is the only approved medical treatment for moderate to severe HS. It is based on two similarly designed (PIONEER I and PIONEER II), Phase 3 multicenter trials of adalimumab for HS. Kimball, A., et al., New Eng. J. Med., 375(5), pp. 422-434 (2016). A total of 307 patients were enrolled in PIONEER I, and 326 were enrolled in PIONEER II. Clinical response rates [hidradenitis suppurativa clinical response (HiSCR): defined as at least a 50% reduction from baseline in the abscess and inflammatory nodule count, with no increase in abscess or draining fistula counts] at Week 12 were significantly higher for the groups receiving adalimumab weekly than for the placebo groups: 41.8% versus 26.0% in PIONEER I (P=0.003) and 58.9% versus 27.6% in PIONEER II (P<0.001). Patients receiving adalimumab had significantly greater improvement than the placebo groups in rank-ordered secondary outcomes (lesions, pain, and the modified Sartorius score for disease severity) at Week 12 in PIONEER II only. Kimball, A., et al., ibid. The main difference between the study designs is that in PIONEER I, patients receiving oral antibiotic agents for HS were required to stop treatment for at least 28 days before baseline; in PIONEER II, patients were allowed to continue treatment with antibiotics (tetracycline class) in stable doses.

Thus, a significant number of patients (˜40%) with moderate to severe HS did not respond to treatment with adalimumab, and therefore there is still an unmet need for an effective, safe, and well tolerated treatment in patients with moderate to severe HS. Disclosed herein is the discovery that compounds and analogues which inhibit certain kinases such as JAK1 and Tyk2 are useful for treating HS. Accordingly, described herein are methods of reducing the severity of HS symptoms in a human subject. These methods can include the step of administering to the subject a pharmaceutical composition comprising such compounds that is effective to reduce the number and/or size of inflammatory lesions (e.g., nodule. abscesses. or draining fistulas), prevent their progression, reduce the pain caused thereby, or delay further lesion development.

### SUMMARY OF THE DISCLOSURE

The present disclosure provides a method for treating hidradenitis suppurativa in a subject to achieve a reduction in flare and elevated levels of HiSCR response.

The present method comprises administering to the subject in need thereof certain compounds disclosed herein that inhibit JAKs, such as JAK1 and Tyk2, in a particular dosage and/or in a particular methodology or treatment regime.

In some embodiments, the disclosure provides a method for treating hidradenitis suppurativa in a subject, the method comprising administering to the subject in need thereof, [(1S)-2,2-difluorocyclo-propyl][(1R,5S)-3-{2-[(1-methyl-1H-pyrazol-4-yl)amino]pyrimidin-4-yl}-3,8-diazabicyclo-[3.2.1]oct-8-yl]methanone or a pharmaceutical salt thereof about 50 mg to about 300 mg per day total and preferably about 100 mg to about 240 mg per day total taken over a period of a day for about 16 weeks or more.

In a further aspect the disclosure provides a method for treating hidradenitis suppurativa in a subject, the method comprising administering to the subject in need thereof, [(1R)-2,2-difluorocyclo-propyl][(1R,5S)-3-{2-[(1-methyl-1H-pyrazol-4-yl)amino]pyrimidin-4-yl}-3,8-diazabicyclo-[3.2.1]oct-8-yl]methanone at a dose of about 50 mg to about 300 mg per day total and preferably about 100 mg to about 240 mg per day total taken over a period of a day for about 16 weeks or more.

Other embodiments of the disclosure include daily oral dosage regimes having the foregoing compounds or pharmaceutically acceptable salts thereof at the disclosed daily amounts. Still other embodiments are compositions having the foregoing compounds or pharmaceutically acceptable salts thereof and one or more pharmaceutically acceptable excipients.

Clinical benefit of the treatment according to the disclosure can be measured, for example by hidradenitis suppurativa clinical response score (HiSCR).

In some embodiments, the JAK inhibitor effectively improves the HiSCR.

Also provided is the use of a JAK inhibitor in the manufacture of a medicament for use in a method of treating and preventing hidradenitis suppurativa in a subject, as described herein. The present disclosure will be further understood from the following description given by way of example only. While the present disclosure is not so limited, an appreciation of various aspects of the disclosure will be gained through the following discussion and the examples.

As used herein, “subject” refers to mammals, companion animals or livestock animals. Mammals are inclusive of humans.

The term “companion animal” or “companion animals” refers to animals kept as pets or household animals. Examples of companion animals include dogs, cats, and rodents including hamsters, guinea pigs, gerbils and the like, rabbits, ferrets, and birds.

The term “livestock” refers to animals reared or raised in an agricultural setting to make products such as food or fiber, or for their labor. In some embodiments, livestock are suitable for consumption by mammals, for example humans. Examples of livestock animals include cattle, goats, horses, pigs, sheep, including lambs, and rabbits, as well as birds, such as chickens, ducks, and turkeys.

The term “treating” or “treatment” means an alleviation of symptoms associated with a disease, disorder or condition, or halt of further progression or worsening of those symptoms. Depending on the disease and condition of the patient, the term “treatment” as used herein may include one or more of curative, palliative, and prophylactic treatment. Treatment can also include administering a pharmaceutical formulation of the present disclosure in combination with other therapies.

The term “therapeutically-effective” indicates the capability of an agent to prevent or improve the severity of a disorder while avoiding adverse side effects typically associated with alternative therapies. The phrase “therapeutically-effective” is to be understood to be equivalent to the phrase “effective for the treatment, prevention, or amelioration”, and both are intended to qualify the amount of each agent for use in the combination therapy which will achieve the goal of improvement in the severity of disease, or pain or other symptom thereof, and the frequency of incidence over treatment of each agent by itself, while avoiding adverse side effects typically associated with alternative therapies.

“Pharmaceutically acceptable” means suitable for use in a “subject.”

### BRIEF DESCRIPTION OF THE DRAWINGS

Embodiments of the present disclosure are described herein with reference to the following figures.

FIG. 1 is a schematic representation of the methodology of the study carried out in Example 9.

FIG. 2 is a plot depicting HiSCR response for Example 9 versus a placebo.

FIG. 3 is a forest plot of several clinical variables for Example 9 versus placebo.

FIG. 4 is a collection of bar graphs for HISCR response at week 16 by baseline Hurley Stage for Example 9 versus placebo.

FIG. 5 is a Kaplan Meier Plot of time to first flare (FAS) for Example 9.

### DETAILED DESCRIPTION OF THE DISCLOSURE

The present disclosure is related to a method for treating hidradenitis suppurativa in a subject, the method comprising administering to the subject in need thereof compounds that inhibit certain JAKs, such as JAK1 and Tyk2. The present disclosure further provides pharmaceutical compositions comprising such inhibitors. Accordingly, the present disclosure provides a method for treating hidradenitis suppurativa in a subject having lesions associated with hidradenitis suppurativa, the method comprising the step of administering to the subject in need thereof a compound of [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone or a pharmaceutically acceptable salt thereof. The disclosure further provides the method wherein said salt is the p-toluenesulfonic acid salt.

The disclosure also provides said method, wherein said compound is [(1R)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone or a pharmaceutically acceptable salt thereof.

The disclosure also provides said method, wherein the subject's HiSCR is improved after administration of the compound.

The disclosure also provides said method, wherein the median size of the subject's hidradenitis suppurativa lesions is reduced after administration of the pharmaceutical composition.

The disclosure also provides said method, wherein the subject's pain associated with the subject's hidradenitis suppurativa lesions is reduced after administration of the compound.

The disclosure also provides said method, wherein the subject's time to develop new hidradenitis suppurativa lesions is increased after administration of the compound.

The disclosure also provides said method, wherein the subject's incidence of flares is reduced.

The disclosure further provides a pharmaceutical or a veterinary composition comprising any compound set forth above, or a pharmaceutically acceptable salt thereof, and a pharmaceutically acceptable carrier, for use in the treatment and prevention of hidradenitis suppurativa.

The disclosure also provides a method for treating hidradenitis suppurativa in a subject having lesions associated with hidradenitis suppurativa, the method comprising the step of administering to the subject in need thereof, a compound which inhibits JAKs, including JAK1 and Tyk2, in an amount effective to treat a symptom of hidradenitis suppurativa in the subject.

The disclosure also provides the method, wherein said effective amount is about 0.01 to about 100 mg/kg of body weight/day, or more preferably about 0.1 to about 10.0 mg/kg, in a single dose or as divided doses administered two, three or four times per day.

The disclosure also provides a method for treating hidradenitis suppurativa in a subject having lesions associated with hidradenitis suppurativa, the method comprising the step of administering to the subject in need thereof, [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone or a pharmaceutically acceptable salt thereof, in an amount effective to treat a symptom of hidradenitis suppurativa in the subject.

The disclosure also provides the method, wherein said effective amount is about 0.01 to about 100 mg/kg of body weight/day, or more preferably about 0.1 to about 10.0 mg/kg, in a single dose or as divided doses administered two, three or four times per day. The disclosure also provides the method, wherein said effective amount is about 45 mg administered QD.

The disclosure also provides the method, wherein the salt is the p-toluenesulfonic acid salt.

The disclosure also provides the method, wherein said effective amount is about 0.01 to about 100 mg/kg of body weight/day, or more preferably about 0.1 to about 10.0 mg/kg, in a single dose or as divided doses administered two, three or four times per day. The disclosure also provides the method, wherein said effective amount is about 400 mg administered QD.

In therapeutic use for treating disorders in a mammal, a compound of the present disclosure or its pharmaceutical compositions can be administered orally, parenterally, topically, rectally, transmucosally, or intestinally. Parenteral administrations include indirect injections to generate a systemic effect or direct injections to the afflicted area. Topical administrations include the treatment of skin or organs readily accessible by local application, for example, eyes or ears. It also includes transdermal delivery to generate a systemic effect. The rectal administration includes the form of suppositories. The preferred routes of administration are oral, topical, and parenteral.

Pharmaceutical compositions of the present disclosure may be manufactured by methods well known in the art, e.g., by means of conventional mixing, dissolving, granulation, dragee-making, levigating, emulsifying, encapsulating, entrapping, lyophilizing processes, or spray drying.

Pharmaceutical compositions for use in accordance with the present disclosure may be formulated in a conventional manner using one or more pharmaceutically acceptable carriers comprising excipients and auxiliaries, which facilitate processing of the active compound into preparations, which can be used pharmaceutically. Proper formulation is dependent upon the route of administration chosen. Pharmaceutically acceptable excipients and carriers are generally known to those skilled in the art and are thus included in the instant disclosure. Such excipients and carriers are described, for example, in Remington's Pharmaceutical Sciences, Mack Pub. Co., New Jersey (1991). The formulations of the disclosure can be designed to be short-acting, fast-releasing, long-acting, and sustained-releasing. Thus, the pharmaceutical formulations can also be formulated for controlled release or for slow release.

Pharmaceutical compositions suitable for use in the present disclosure include compositions wherein the active ingredients are contained in an amount sufficient to achieve the intended purpose, i.e., control or the treatment of HS. More specifically, a therapeutically effective amount means an amount of compound effective to prevent, alleviate or ameliorate symptoms/signs of the disease or prolong the survival of the subject being treated.

The quantity of active component, which is the compound of this disclosure, in the pharmaceutical composition and unit dosage form thereof, may be varied or adjusted widely depending upon the manner of administration, the potency of the particular compound and the desired concentration. Generally, the quantity of active component will range between 0.01% to 99% by weight of the composition.

Generally, a therapeutically effective amount of dosage of active component will be in the range of about 0.01 to about 100 mg/kg of body weight/day, preferably about 0.1 to about 10 mg/kg of body weight/day, more preferably about 0.3 to 3 mg/kg of body weight/day, even more preferably about 0.3 to 1.5 mg/kg of body weight/day. It is to be understood that the dosages may vary depending upon the requirements of each subject and the severity of the disorders or diseases being treated.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

In a preferred embodiment, the lesions of hidradenitis suppurativa are treated by administering to a human subject or patient in need thereof a compound of [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone (also referred to as brepocitinib or PF-06700841) or a pharmaceutically acceptable salt thereof. The compound is administered in a dosage of about 50 mg to about 300 mg per day total and preferably about 100 mg to about 240 mg per day total taken over a period of a day for about 16 weeks or more. A particularly preferred dosage is about 25 mg to about 100 mg four times per day for about 16 weeks or more. A most preferred dosage is about 45 mg four times per day for about 16 weeks or more. The amount administered preferably is effective to enable a Hurley 2 subject or patient to achieve a HiSCR response of ≥50% within 16 weeks of initiation of administration. The amount administered preferably is effective to enable a Hurley 3 subject or patient to achieve a HiSCR response of ≥30% within 16 weeks of initiation of administration. The amount administered preferably is effective to enable a subject or patient to achieve a probability of flare of ≤20% for up to 16 weeks after initiation of administration. Oral administration is preferred.

Suitable agents for use in combination therapy with a compound set forth herein, or a pharmaceutically acceptable salt thereof, or pharmaceutically acceptable solvate of said compound or salt, particularly in the treatment of the disease, include: a 5-lipoxygenase activating protein (FLAP) antagonist; a leukotriene antagonist (LTRA) such as an antagonist of LTB₄, LTC₄, LTD₄, LTE₄, CysLT₁ or CysLT₂, e.g., montelukast or zafirlukast; a histamine receptor antagonist, such as a histamine type 1 receptor antagonist or a histamine type 2 receptor antagonist, e.g., loratidine, fexofenadine, desloratidine, levocetirizine, methapyrilene or cetirizine; an α1-adrenoceptor agonist or an α2-adrenoceptor agonist, e.g., phenylephrine, methoxamine, oxymetazoline or methylnorephrine; a muscarinic M3 receptor antagonist, e.g. tiotropium or ipratropium; a dual muscarinic M3 receptor antagononist/β2 agonist; a PDE inhibitor, such as a PDE3 inhibitor, a PDE4 inhibitor or a PDE5 inhibitor, e.g., theophylline, sildenafil, vardenafil, tadalafil, ibudilast, cilomilast or roflumilast; sodium cromoglycate or sodium nedocromil; a cyclooxygenase (COX) inhibitor, such as a non-selective inhibitor (e.g., aspirin or ibuprofen) or a selective inhibitor (e.g. celecoxib or valdecoxib); a glucocorticosteroid, e.g., fluticasone, mometasone, dexamethasone, prednisolone, budesonide, ciclesonide or beclamethasone; an anti-inflammatory monoclonal antibody, e.g., infliximab, adalimumab, tanezumab, ranibizumab, bevacizumab or mepolizumab; a β2 agonist, e.g., salmeterol, albuterol, salbutamol, fenoterol or formoterol, particularly a long-acting β2 agonist; an intigrin antagonist, e.g., natalizumab; an adhesion molecule inhibitor, such as a VLA-4 antagonist; a kinin B₁ or B₂ receptor antagonist; an immunosuppressive agent, such as an inhibitor of the IgE pathway (e.g., omalizumab) or cyclosporine; a matrix metalloprotease (MMP) inhibitor, such as an inhibitor of MMP-9 or MMP-12; a tachykinin NK₁, NK₂ or NK₃ receptor antagonist; a protease inhibitor, such as an inhibitor of elastase, chymase or catheopsin G; an adenosine Ata receptor agonist; an adenosine A_{2b} receptor antagonist; a urokinase inhibitor; a dopamine receptor agonist (e.g., ropinirole), particularly a dopamine D2 receptor agonist (e.g., bromocriptine); a modulator of the NFκB pathway, such as an IKK inhibitor; a further modulator of a cytokine signaling pathway such as an inhibitor of syk kinase, p38 kinase, SPHK-1 kinase, Rho kinase, EGF-R or MK-2; a mucolytic, mucokinetic or anti-tussive agent; an antibiotic; an antiviral agent; a vaccine; a chemokine; an epithelial sodium channel (ENaC) blocker or Epithelial sodium channel (ENaC) inhibitor; a nucleotide receptor agonist, such as a P2Y2 agonist; a thromboxane inhibitor; niacin; a 5-lipoxygenase (5-LO) inhibitor, e.g., Zileuton; an adhesion factor, such as VLAM, ICAM or ELAM; a CRTH2 receptor (DP₂) antagonist; a prostaglandin D₂ receptor (DP₁) antagonist; a haematopoietic prostaglandin D2 synthase (HPGDS) inhibitor; interferon-β; a soluble human TNF receptor, e.g., Etanercept; a HDAC inhibitor; a phosphoinositotide 3-kinase gamma (PI3Kγ) inhibitor; a phosphoinositide 3-kinase delta (PI3Kδ) inhibitor; a CXCR-1 or a CXCR-2 receptor antagonist; an IRAK-4 inhibitor; and, a TLR-4 or TLR-9 inhibitor, including the pharmaceutically acceptable salts of the specifically named compounds and the pharmaceutically acceptable solvates of said specifically named compounds and salts.

Pharmaceutically acceptable excipients can include, but are not limited to, binders, lubricants, glidants, inert diluents, preservatives, disintegrants, and dispersing agents. Tablets and other solid dosage forms, such as, but not limited to, capsules, pills, powders, and granules, can include coatings, such as enteric coatings.

### Chemical Synthesis

The compounds of the disclosure may be prepared by any method known in the art. In particular, the compounds of the disclosure can be prepared by the procedures described by reference to the prior art references in which they are disclosed.

For those compounds that inhibit Tyk2 and JAK1 specifically, including [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone, preparative methods are disclosed in U.S. Pat. No. 9,663,526, the contents of which are incorporated herein in their entirety.

The contents of U.S. Provisional Application No. 62/899,133, filed Sep. 11, 2019, referenced in PCT Publication No. 2021/048736, are incorporated herein in their entirety.

### EXAMPLES

The following non-limiting examples are presented merely to illustrate the present disclosure. The skilled person will understand that there are numerous equivalents and variations not exemplified but which still form part of the present teachings.

### Example 1

### Hidradenitis Suppurativa Clinical Response (HiSCR)

This study provided data on efficacy, safety, tolerability, and pharmacokinetics of the therapeutic agents being examined in the oral treatment of moderate to severe HS. The study had a maximum duration of approximately 26 weeks. This included an up-to-6-week Screening Period, a 16-week Dosing Period and a 4-week Follow-up Period. The study enrolled a total of approximately 192 participants (expected to provide approximately 156 completers). Following the screening period, participants who meet eligibility criteria at the baseline visit were randomly assigned to receive 1 of 6 treatments. One oral dose level of each therapeutic agent, one of which was [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone (45 mg QD), or matching placebo in a 3:1 ratio was investigated. For analysis, placebo groups were combined to yield final therapeutic agent:placebo ratio of 1:1:1:1 for each therapeutic agent and pooled placebo. No more than 30% of enrolled participants were inadequate anti-TNF responders. Participants were stratified according to whether they were an inadequate anti-TNF responder or not.

Additionally, no more than 20% of enrolled participants entered the study on a background of concomitant oral antibiotic therapy for treatment of HS; the dosing regimen (dose and frequency) must have been stable for at least 8 weeks (56 days) prior to the baseline (Day 1) visit and remained stable throughout study participation. Antibiotics taken on an “as needed” (PRN) basis were not considered a stable dose. Participants were stratified according to whether they were on a background of concomitant antibiotic therapy or not. The chronic toxicology package for each asset supported the planned study treatment duration of 16 weeks. Hidradenitis suppurative clinical response, the primary endpoint used, is defined as:

at least a 50% reduction in the total abscess and inflammatory nodule (AN) count relative to baseline, and no increase in abscess count, and no increase in draining fistula count.

### Example 2

### Lesion Counts

The number of inflammatory and non-inflammatory nodules, abscesses, draining and non-draining fistulas, and hypertrophic scars, as well as the physical location (right/left axilla, right/left inframammary, intermammary, right/left buttock, right/left inguino-crural fold, perianal, perineal, other) were assessed according to the standard of the art.

### Example 3

### Abscess Count

Number of abscesses (fluctuant, with or without drainage, tender or painful) were counted in each of the regions as defined above.

### Example 4

### Inflammatory Nodule Count

Number of inflammatory nodules (tender, erythematous, pyogenic granuloma lesion) were counted in each of the regions as defined above.

### Example 5

### Fistula Count

Number of fistula (sinus tracts, with communications to skin surface, draining purulent fluid) were counted in each of the regions as defined above.

### Example 6

### Hurley Staging

Hurley staging is defined as follows:
Stage I: Abscess formation, single or multiple, without sinus tracts and cicatrization (scarring).
Stage II: One or more widely separated recurrent abscesses with tract formation and cicatrization (scars).
Stage III: Multiple interconnected tracts and abscesses across the entire area, with diffuse or near diffuse involvement.

Hurley staging was performed according to the standard of the art.

### Example 7

### Modified Sartorius Scale

The Modified Sartorius score was calculated by counting lesions in the following 12 anatomic regions: left axilla, right axilla, left sub/inframammary area, right sub/inframammary area, intermammary area, left buttock, right buttock, left inguino-crural fold, right inguino-crural fold, perianal area, perineal area, other. For each anatomic region, calculate the regional Sartorius score as follows:

Anatomic region involved: 3 points per region involved (i.e., any lesion count in this anatomic region >0; otherwise, 0 points).

Number and scores of lesions (abscesses, nodules, fistulas, scars): 2 points for each nodule (inflammatory and non-inflammatory), 4 points for each abscess, 4 points for each fistula (draining and non-draining), 1 point for each hypertrophic scar, 1 point for each “other”.

Longest distance between 2 relative lesions (i.e., 0 if no active lesion; 2 if longest distance between 2 relevant lesions or size <50 mm; 4 if longest distance between 2 relevant lesions or size ≥50 mm and <100 mm; 6 if longest distance between 2 relevant lesions or size ≥100 mm.

Lesions clearly separated by normal skin in each region: If all lesions clearly separated by normal appearing skin, 0 points; 6 points if otherwise.

The total Modified Sartorius score is the sum of all of the 12 regional scores.

### Example 8

### Erythema Assessment

The overall degree of erythema was assessed for each anatomic region affected by HS using a four-point ordinal scale ranging between 0 (no redness), 1 (faint but discernible pink coloration), 2 (moderate red coloration), or 3 (very red or bright red coloration).

### Example 9 and Comparative Example 1

### Summary

The above Examples were a phase 2A double-blind, parallel group study assessing the efficacy of PF-06700841, a dual inhibitor of human tyrosine kinase 2 (TYK2) and Janus kinase 1 (JAK1), compared to Placebo in human patients with moderate to severe hidradenitis suppurativa (HS). PF-06700841 met the predefined primary efficacy criteria (multiplicity adjusted p-value <0.1).

Tests carried out on PF-06700841 constitute Example 9. PF-06700841 refers to brepocitinib, (1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone.

Tests carried out with placebo constitute Comparative Example 1.

Placebo-adjusted percentage of HiSCR rate (50% reduction from baseline in abscess and inflammatory nodule (AN) count, with no increase in abscess and draining fistula count from baseline) at week 16 for PF-06700841 was 18.7 (p=0.0298). The PF-06700841 group (along with the placebo group) had relatively higher response rate among moderate HS participants (Hurley Stage II at the baseline), with respect to the primary endpoint HiSCR response rate at week 16. Statistically significant reduction in percentage of participants experiencing at least one flare (defined as at least a 25% increase in AN count with a minimum increase of 2 relative to baseline) by week 16 from placebo was observed in the PF-06700841 (δ=−22.3 ρ=0.006) group. Pain is an important symptom in participants with HS. For the percentage of NRS30 response rate (≥30% reduction and ≥1-unit reduction from baseline in PGA-Skin Pain numeric rating scale (NRS30) at worst) among participants with baseline score 3 at week 16, the difference from placebo for PF-06700841 was 6.5 (p=0.265). Overall consistent and robust efficacy was observed across primary and secondary endpoints for PF-06700841. PF-06700841 appeared generally safe and well tolerated. The majority of the treatment-emergent adverse events (TEAEs) reported were mild and moderate. There were no deaths or cases of herpes zoster/simplex in the study. Based on preliminary population PK analysis the expected exposures in HS appear to be achieved when compared to either healthy or other patient populations for the compound.

### Test Design

This was a randomized, double-blind, parallel-group multi-center study that enrolled approximately 192 participants with moderate to severe HS (with approximately 156 completers). Participants were randomized to PF-06700841 (along with two other treatments studied contemporaneously) and matching placebo in a 3:1 ratio. Each randomized participant was followed for a 16-week dosing period and a 4-week follow-up period. For analysis, placebo groups were combined to yield a 1:1:1:1 ratio for each treatment and pooled placebo. No more than 30% participants were enrolled with inadequate anti-TNF response and no more that 20% of the participants were on background antibiotic therapy, and stratified randomization strategy was adopted for this study. Efficacy data from all randomized participants until week 16 are in the scope of this report.

### Primary Objective and Primary Endpoint

The primary objective of the study was to evaluate the efficacy and safety of PF-06700841 vs placebo in participants with HS. The primary efficacy endpoint was percentage of participants with HiSCR response (50% reduction from baseline in abscess and inflammatory nodule (AN) count, with no increase in abscess and draining fistula count from baseline) at week 16.

### Secondary Objective and Endpoints

Percentage of participants who experienced at least one flare event (defined as at least a 25% increase in AN count with a minimum increase of 2 relative to baseline after week 4) by week 16.
Percentage of participants achieving total AN count of 0, 1 or 2 at week 16.
Percentage of participants achieving ≥30% reduction and ≥1-unit reduction from baseline in PGA-Skin Pain numeric rating scale (NRS30) at worst at week 16 among participants with baseline NRS≥3.
Percent change from baseline in International Hidradenitis Suppurativa Severity Score System (IHS4) at week 16. This IHS4 score is a weighted scoring system and calculated by the number of nodules multiplied by 1+the number of abscesses multiplied by 2+the number of draining tunnels (fistulas/sinuses) multiplied by 4. Mild HS is defined by IHS4 11 points.

The primary and secondary efficacy analysis were performed using data from all participants, but treatment effect was also assessed among participants with different disease severity based on baseline Hurley Stage. The study population consists of 67% Stage II (recurrent abscesses with tract formation and cicatrization, single or multiple, widely separated lesions) participants and 33% Stage III participants (multiple interconnected tracts and abscesses across the entire area, with diffuse or near diffuse involvement).

The safety and tolerability of PF-06700841 over time was assessed using incidence of treatment-emergent adverse events (TEAEs), vital signs, incidence of specific clinical laboratory abnormalities including but not limited to hemoglobin, neutrophils, platelets, lymphocytes, lipids, eGFR, liver function tests (LFTs) and CPK.

### Analysis Population and Methodology

All participants who received at least one dose of randomized study medication and have a baseline are included in the full analysis set (FAS), the population for the efficacy analysis. The safety analysis set (SAS) includes all participants who received at least one dose of investigational product. The HiSCR response data at week 16 comparing active treatment group and placebo group was to be analyzed using the Cochran Mantel Hanzel (CMH) test with Minimum Risk (MR) weight strategy after treating missing data as non-responders, adjusting for the stratification factor of prior anti-TNF failure status. Due to very small number of participants with concomitant use of antibiotics status at baseline in some subgroups, this was not included as stratum factor in the model. Hochberg's step-up procedure was employed to address any multiplicity arising from the analysis of 3 different treatments within the same study and overall familywise error rate was controlled at 0.1 level. All the continuous secondary endpoints were analyzed using ANCOVA model, where missing observation were imputed using multiple imputation (MI) method.

### Pharmacokinetics

Blood was collected to obtain the PK samples at pre-dose, Weeks 1, 2, 4, 6, 8, and 16, and at 0.5-, 1-, 2-, and 4-hours post-dose on Week 8. The available data was analyzed using population PK modeling.

### Participant Population, Baseline and Disease Characteristics

There were 359 participants screened (67% moderate and 33% severe), and HS participants were enrolled into the study in over 60 sites across 3 countries (United States, Canada, and Australia). Data from these moderate to severe participants were included in the efficacy analysis set (FAS) and safety analysis set (SAS). The patient demographic and baseline characteristics (Table 1) are similar and generally well balanced. The treated participants had an average total AN count baseline score of 13 (SD=10.9). The majority (77.8%) of the participants were females. The average total AN count was 12 (SD=10.0) for PF-06700841. At baseline, there were participants with anti-TNF Inadequate response, and participants were on a background of concomitant oral antibiotic therapy. The treatment groups appeared to be comparable with respect to age, gender, total AN count, fistula count, IHS4 score and pain score at baseline.

FIG. 2 is a plot depicting HiSCR response for Example 9 versus the placebo. The plot shows an estimate and 90% confidence interval (CI) for percentage of participants with HiSCR response over time [Placebo (Red), PF-06700841 (Yellow)]. The yellow plot exhibited the highest response at week 16. The red plot exhibited the lowest response at week 16.

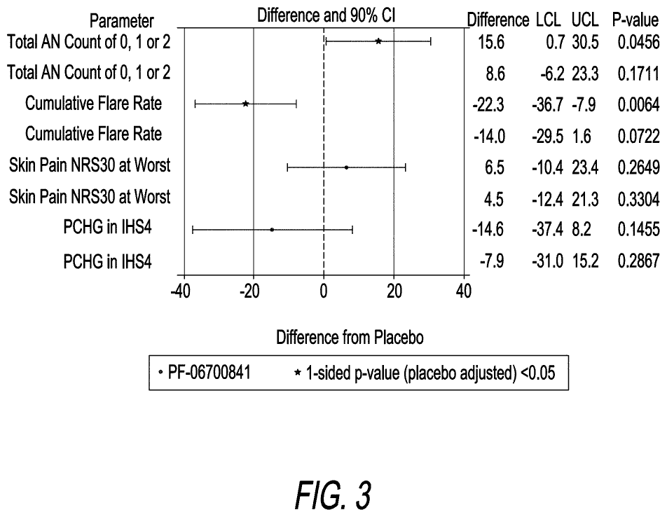

FIG. 3 is a forest plot of AN count for Example 9 versus placebo. A forest plot of estimates (90% CI) for differences is compared to placebo at week 16 for a percentage of participants experiencing at least one flare event, AN count at 0, 1 or 2, NRS-30 (30% reduction in weekly average pain score, with baseline score of at least 3), and a percent change from baseline in IHS4 score at week 16, PF-06700841 (yellow).

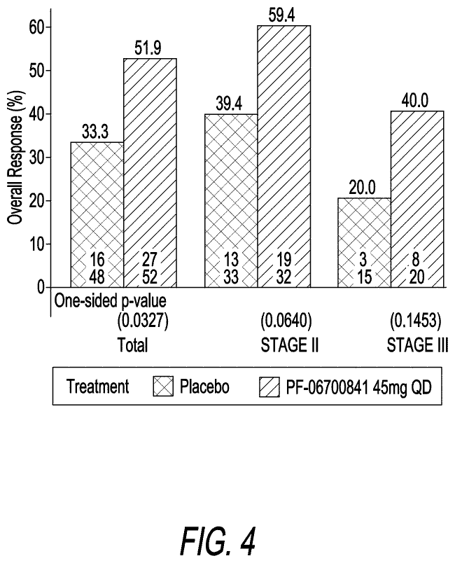

FIG. 4 is a collection of bar graphs for HISCR response at week 16 by baseline Hurley Stage for Example 9 versus placebo. The percentage of participants achieving HiSCR response at week 16 by baseline Hurley Stage (FAS, NRI) is shown.

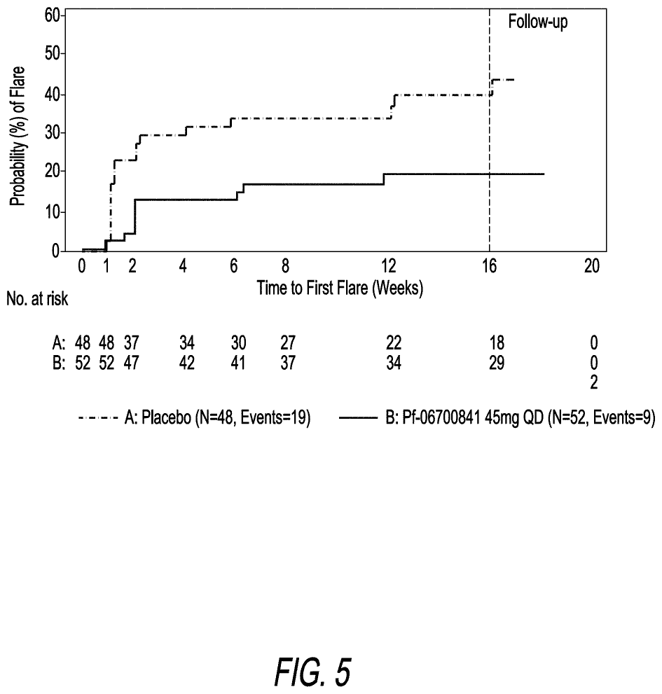

FIG. 5 is a Kaplan Meier Plot comparing time to first flare (FAS) for each of placebo (red) and PF-06700841 (yellow). The placebo plot depicts the highest probability of flare at 16 weeks. The PF-06700841 plot depicts the lowest probability of flare at 16 weeks.

TABLE 1
Baseline and Disease Characteristics

| | Placebo | PF-06700841 |
|---|---|---|
| | (N = 48) | (N = 52) |
| Age (years): mean (SD) | 37 (9.0) | 38 (11.8) |
| Gender (Female): n (%) | 42 (87.5) | 40 (76.9) |
| Race: n (%) | | |
| White | 32 (66.7) | 26 (50.0) |
| Black | 13 (27.1) | 24 (46.2) |
| Asian | 2 (4.2) | 1 (1.9) |
| American Indian/Alaska Native | 1 (2.1) | 1 (1.9) |
| Multiracial (Asian, White) | 0 | 0 |
| Not reported | 0 | 0 |
| BMI: mean (SD) | 35.12 (7.31) | 36.28 (8.64) |
| Disease Duration (years): mean (SD) | 13.6 (8.67) | 12.2 (8.65) |
| Anti-TNF Inadequate Responder: n(%) | | |
| Yes | 10 (20.8) | 12 (23.1) |
| No | 38 (79.2) | 40 (76.9) |
| Concomitant Use of | | |
| Antibiotics Treatment: n (%) | | |
| Yes | 7 (14.6) | 7 (13.5) |
| No | 41 (85.4) | 45 (86.5) |
| Hurley Stages: n (%) | | |
| II | 33 (66.8) | 32 (61.5) |
| III | 15 (31.3) | 20 (38.5) |
| Total AN Count | | |
| Mean (SD) | 12 (8.1) | 13 (11.4) |
| Median (Range) | 9 (4, 39) | 9 (2, 58) |
| Draining Fistula Count | | |
| Mean (SD) | 2 (2.8) | 3 (3.1) |
| Median (Range) | 1 (0, 14) | 2 (0, 16) |
| IHS4 | | |
| Mean (SD) | 23 (16.0) | 26 (23.8) |
| Median (Range) | 17 (4, 78) | 21 (4, 128) |
| Skin Pain NRS at Worst | | |
| Mean (SD) | 5.8 (2.45) | 5.6 (2.54) |
| Median (Range) | 5.7 (1, 10) | 5.2 (0.8, 10) |
| ≥3, n (%) | 41 (85.4) | 42 (80.8) |

### Efficacy

PF-06700841 met the pre-specified efficacy criteria for the primary endpoint. The stratified CMH test estimate of the differences from placebo in HiSCR response rate (90% CI) at week 16 was 18.7 [90% CI=(2.7, 34.6)]. The results from the sensitivity analysis (unstratified analysis using Chan and Zhang (1999) exact method) are similar to the primary analysis.

TABLE 2
Percentage of Participant Achieving HISCR Response
at Week 16 (FAS, NR1)

| | Placebo | PF-06700841 |
|---|---|---|
| | (N = 48) | (N = 52) |
| Overall Response n/N (%) | 16/48 (33.3) | 27/52 (51.9) |
| 90% CI | (22.2, 45.5) | (39.7, 64.0) |
| Difference from Placebo | | |
| Estimate (SE) | | 18.7 (9.71) |
| 90% CI | | (2.7, 34.6) |
| One-Side p-value | | 0.0298 |
| Hochberg adjusted p-value | | 0.0893 |

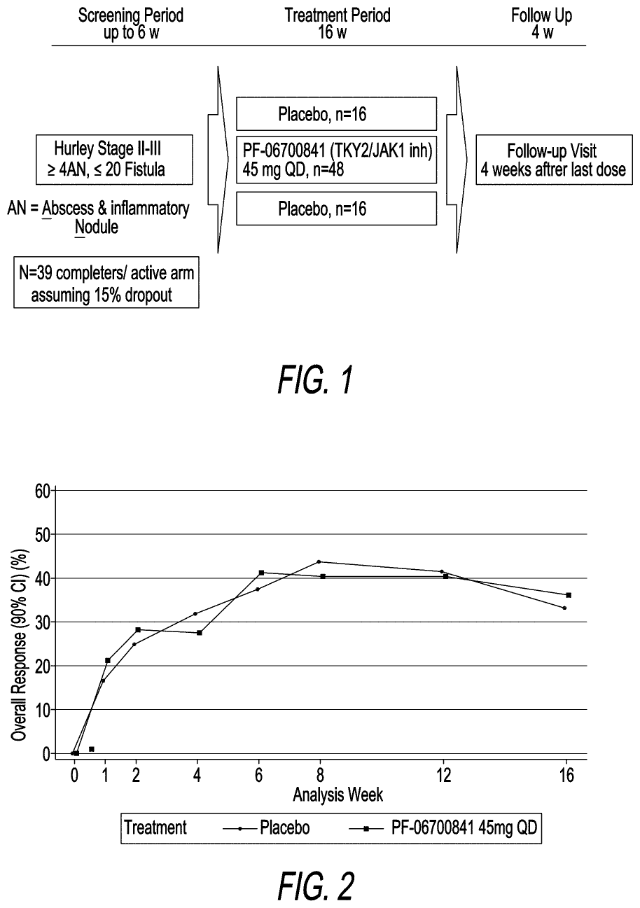

No statistical separation of HiSCR response rate from the placebo was observed by week 12 for all three compounds (FIG. 1). Participants with moderate disease severity at the baseline (Hurley Stage II) had higher response rate with all three active treatments and with Placebo compared to the participants with severe HS (Hurley Stage III at baseline). Numerically higher response rate (HiSCR response rate=59.4%) in the primary endpoint at week 16 was observed among Hurley Stage II participants, compared to Hurley Stage III participants (40%) and overall population (51.9%) with PF-06700841 treatment (FIG. 4).

Results from Secondary Endpoints:
1. percentage of participants achieving at least one flare event by week 16
2. percentage of participants achieving total AN count 0, 1, or 2 at week 16
3. percentage of participants achieved NRS30 response; and
4. percentage change from baseline in IHS4 at week 16 for PF-06700841 are provided in FIG. 3. Flare event (defined as at least a 25% increase in AN count with a minimum increase of 2 relative to baseline 25%) was a pre-specified secondary endpoint to this study and statistically significant reduction in flare event rate (at least one flare event by week 16) was observed with PF-06700841 (δ=−22.3, p=0.006) treatment group. Treatment group had significant reduction in time to first flare represented in FIG. 5. Cox proportional model estimates the hazard ratio for time to first flare as 0.4 for PF-06700841. Clinically significant reduction in AN count was demonstrated by achieving AN count of 0, 1 or 2 by end of 16 weeks of the treatment in this study. More participants (15.6%) in the PF-06700841 treatment group compared to placebo (p=0.046) achieved total AN count of 0, 1 or 2 at week 16.

The Patient's Global Assessment of Skin Pain Numeric Rating Scale (NRS) was used to assess the worst skin pain and the average skin pain due to HS. The assessments were completed on a daily diary by participants before they went to bed and responded to the items based on a recall period of the last 24 hours. Significant amounts of data (%) with respect to this endpoint were missing. The missing data were imputed using LOCF (last observation carried forward) method. None of the compounds differentiated from Placebo on Skin Pain NRS30 at week 16 (FIG. 2). The difference in Skin Pain NRS30 at week 16 from Placebo for PF-06700841 was 6.5 (p=0.265). Results from other patient-reported outcome endpoints such as change from baseline in Dermatology Life Quality Index (DLQI) over time and change from baseline in domains of the Short Form Health Survey version 2 Acute (SF36v2 Acute) over time were consistent with other endpoints. The difference from Placebo in percent change from baseline at week 16 for the IHS4 score in PF-06700841 treatment group was −14.6 (p=0.145).

It should be understood that the foregoing description is only illustrative of the present disclosure. Various alternatives and modifications can be devised by those skilled in the art without departing from the present disclosure. Accordingly, the present disclosure is intended to embrace all such alternatives, modifications and variances that fall within the scope of the appended claims.

1. A method for treating hidradenitis suppurativa in a human subject having lesions associated with hidradenitis suppurativa, the method comprising the step of administering to the subject in need thereof a compound of [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone; or a pharmaceutically acceptable salt thereof, wherein the compound is administered in a dosage of about 50 mg to about 300 mg per day total taken over a period of a day.

2. A method for treating hidradenitis suppurativa in a human subject having lesions associated with hidradenitis suppurativa, the method comprising the step of administering to the subject in need thereof a compound of [(1R)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone; or a pharmaceutically acceptable salt thereof, wherein the compound is administered in a dosage of about 50 mg to about 300 mg per day total taken over a period of a day.

3. The method of claim 1, wherein the salt is the p-toluenesulfonic acid salt.

4. The method of claim 1, wherein the compound is administered in a dosage of about 100 mg to about 240 mg total taken over a period of a day.

5. The method of claim 1, wherein the compound is administered in a dosage of about 25 mg to about 100 mg four times per day.

6. The method of claim 1, wherein the compound is administered in a dosage of about 45 mg four times per day.

7. The method of any of claim 1, wherein the subject is a Hurley 2 subject, wherein the subject achieves a HiSCR response of ≥50% within 16 weeks of initiation of administration.

8. The method of any of claim 1, wherein the subject is a Hurley 3 subject, wherein the subject achieves a HiSCR response of ≥30% within 16 weeks after initiation of administration.

9. The method of any of claim 1, wherein the subject has a probability of flare is ≤20% for up to 16 weeks after initiation of administration.

10. The method of any of claim 1, wherein the treatment reduces inflammatory nodules, abscess and draining fistulas as measured by the subject's HiSCR score.

11. The method of any of claim 1, wherein the treatment reduces the median size of the subject's hidradenitis suppurativa lesions.

12. The method of any of claim 1, wherein the treatment reduces the subject's pain associated with the subject's hidradenitis suppurativa lesions.

13. The method of any of claim 1, wherein the treatment increases the subject's time to develop new hidradenitis suppurativa lesions.

14. The method of any of claim 1, wherein said administration is in a single dose or as divided doses administered two, three or four times per day.

15. A daily dosage regime for treating hidradenitis suppurativa in a subject having lesions associated with hidradenitis suppurativa, wherein the compound is [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone; or a pharmaceutically acceptable salt thereof, wherein the compound is present in the dosage regime at about 50 mg to about 300 mg.

16. A daily dosage regime for treating hidradenitis suppurativa in a subject having lesions associated with hidradenitis suppurativa, wherein the compound is [(1R)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone; or a pharmaceutically acceptable salt thereof, wherein the compound is present in the dosage regime at about 50 mg to about 300 mg per day total taken over a period of a day.

17. A composition for treating hidradenitis suppurativa in a subject having lesions associated with hidradenitis suppurativa, wherein the composition includes a compound of [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone or a pharmaceutically acceptable salt thereof in an amount about 50 mg to about 300 mg and a pharmaceutically acceptable excipient.

18. A composition for treating hidradenitis suppurativa in a subject having lesions associated with hidradenitis suppurativa, wherein the composition includes a compound of [(1R)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone or a pharmaceutically acceptable salt thereof in an amount about 50 mg to about 300 mg and a pharmaceutically acceptable excipient.
